(19) United States
(12) Patent Application Publication
(10) Pub. No.: US 2025/0090762 A1
(43) Pub. Date: Mar. 20, 2025

(54) ELECTRONIC ENCODER MODULE FOR A DRUG DELIVERY DEVICE AND DRUG DELIVERY DEVICE

(71) Applicant: Sanofi, Paris (FR)

(72) Inventors: Matthias Felber, Wil (CH); Florian Eberli, Wil (CH); Adrian Geiger, Wil (CH)

(21) Appl. No.: 18/727,282

(22) PCT Filed: Jan. 9, 2023
(86) PCT No.: PCT/EP2023/050258

§ 371 Date: Jul. 8, 2024

(30) Foreign Application Priority Data
Jan. 10, 2022 (EP) 22315007.9

(51) Int. Cl. A61M 5/315

(52) U.S. Cl. CPC A61M 5/3155; A61M 5/31568

(57) ABSTRACT

A method of charging an electronic module of a drug delivery device and a respective charging system. An electronic encoder module may comprise at least one sensor unit, a PCBA with a processor configured to control operation of the at least one sensor unit and to process and/or store signals from the at least one sensor unit, a rechargeable battery connected to the PCBA, and a cap-shaped outer shell at least partially encasing the at least one sensor unit, the PCBA and the battery. The outer shell comprises a lateral surface and a proximally arranged top surface, wherein at least two electrical contacts connected to the battery and/or the PCBA are provided on the lateral surface and/or on the proximally arranged top surface.

### CROSS-REFERENCE TO RELATED APPLICATIONS

This is a National Stage Application, filed under 35 U.S.C. § 371, of International Patent Application No. PCT/EP2023/050258, filed on Jan. 9, 2023, which claims priority to European Patent Application No. 22315007.9, filed on Jan. 10, 2022, the disclosures of which are incorporated herein by reference in their entireties.

### TECHNICAL FIELD

The present invention is generally directed to an electronic encoder module and to a drug delivery device with such a module. Further, a method for charging an electronic module of a drug delivery device and a charging system for charging an electronic module of a drug delivery device are disclosed.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament. However, setting the correct dose amount may be difficult or burdensome for, e.g., visually and/or manually impaired patients.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present invention is applicable for disposable and reusable devices.

For such devices the functionality of recording doses that are dialed and delivered from the pen may be of value to a wide variety of device users as a memory aid or to support detailed logging of dose history. Thus, drug delivery devices using electronics are becoming increasingly popular in the pharmaceutical industry as well as for users or patients. For example, a dose recording system is known from WO 2021/116387 A1 comprising a drug delivery device and an electronic module which is removably mechanically coupled to the drug delivery device. The electronic module of this known device is provided with a rechargeable battery (accumulator). Further, WO 2021/233799 A1 discloses a dial grip for an injection device, wherein the dial grip comprises internal electronics with a power source. Electrical contacts are provided on the handling part and the grip top. WO 2019/101962 A1 and WO 2018/0134419 A1 disclose encoder modules for drug delivery devices.

### SUMMARY

It is an object of the present disclosure to further improve user-friendliness of charging of an electronic module used in or with pen-type drug delivery devices.

This object is solved for example by the subject matter defined in the independent claims. Advantageous embodiments and refinements are subject to the dependent claims. However, it should be noted that the disclosure is not restricted to the subject matter defined in the appended claims. Rather, the disclosure may comprise improvements in addition or as an alternative to the ones defined in the independent claims as will become apparent from the following description.

One aspect of the disclosure relates to an electronic encoder module for use with a drug delivery device wherein the electronic encoder module comprises at least one sensor unit, a PCBA with a processor configured to control operation of the at least one sensor unit and to process and/or store signals from the at least one sensor unit, a rechargeable battery (accumulator) connected to the PCBA, and a cap-shaped outer shell at least partially encasing the at least one sensor unit, the PCBA and the battery. The sensor unit may be part of the PCBA or may be connected, e.g., soldered, to the PCBA. The outer shell may comprise a lateral surface, e.g., a substantially cylindrical lateral surface, and a proximally arranged top surface, e.g., a flat or dome-shaped top surface. Charging of the battery may be effected by means of at least two electrical contacts connected to the battery and/or the PCBA which contacts are provided on the lateral surface and/or on the proximally arranged top surface. The electrical contacts are preferably configured to come into abutment with, e.g., spring-loaded or elastic contacts of a charging system, for example inside a charging station, cable connector or storage container. Such external, e.g., visible, contacts have the advantage of providing a simple, reliable, small-size and watertight possibility for charging a battery of an electronic module.

The electrical contacts are made from an electrically conductive material, e.g., a metal. The electrical contacts may comprise at least one ring, at least one ring segment and/or at least one contact area made from an electrically conductive material and being surrounded by a non-conductive material of the outer shell. For example, when formed by a segmented ring or two rings, the contacts may be arranged axially symmetrical with respect to the longitudinal axis of the electronic encoder module. In an example, the electrical contacts are both arranged on the lateral surface of the outer shell. In an alternative example, the electrical contacts are both arranged on the top surface of the outer shell.

Spring contacts or a charging cable comprising supply contacts may be attached to the module by magnets. Further, the contacts may be used as a sensing element for activation of the module, e.g., by means of a finger detection by impedance measurement. In addition or as an alternative, the contacts may be used as a sensing element for detecting when the finger is removed again from the button, e.g., to monitor the holding time after the injection, or for doing plausibility checks related to the activation.

The electrical contacts may be attached to the outer shell by gluing, by press fit and/or by overmolding. Further, the electronic encoder module may comprise a covered state and an uncovered state, wherein in said covered state a cap is attached to said electronic encoder module, and wherein said cap covers at least the electrical contacts of said electronic encoder module provided for charging.

The electronic encoder module may have a battery capable of supplying energy for several days, e.g., at least one week, of use in or with a drug delivery device. The rechargeable battery may be a coin cell, e.g., a NiMH battery, with a charging capacity, e.g., above 5 mAh and/or up to 50 mAh, for example between 6 mAh and 25 mAh. This is suitable for about 6 weeks of battery lifetime. The battery may have a height between 1.5 mm to 4.5 mm which avoids that the module is too bulky. Further, the module may comprise a boost (power) supply unit, i.e., a boost converter unit, configured to provide a constant equal voltage, for example 3.3 V. The boost (power) supply unit may comprise at least one capacitor which can be used as a DC source with an output voltage greater than the DC voltage powering the circuit, e.g., using a battery supplying only 1.2V.

In the electronic encoder module the at least one sensor unit may comprise at least one, e.g., two, light sources, for example IR-LEDs, and at least two optical sensors, for example light detectors. The sensor unit may be configured for detecting a movement of an, e.g., rotatable, encoder element of a drug delivery device if the module is attached to the drug delivery device, wherein the movement is indicative of doses that are dialed and/or delivered from the drug delivery device.

In more detail, a first light source and a first optical sensor may be arranged next to each other and facing towards a sensor side end of a first light pipe, wherein a second light source and a second optical sensor may be arranged next to each other and facing towards a sensor side end of a second light pipe. Further, the light pipes may each have an opposite encoder side end which if occluded by a light reflective material, e.g., of an encoder disk or encoder ring of the drug delivery device, result in a light beam emitted from the respective first or second light source to be detected by the respective first or second optical sensor after being reflected by the encoder element.

The electronic module may be permanently or releasably attached or integrated in a drug delivery device for setting and dispensing variable doses of a liquid drug. For example, the drug delivery device comprises a cartridge containing a liquid drug and a dose setting and drive mechanism which is configured to perform a dose dialing operation for selecting a dose to be delivered by the drug delivery device and a dose delivery operation for delivering the set dose. The electronic module may comprise at least one sensor unit configured to detect operation of the dose setting and drive mechanism and a processor configured to control operation of the at least one sensor and to process and/or store signals from the at least one sensor. For example, the at least one sensor unit may comprise an optical sensor for detecting a dose delivery operation of the dose setting and drive mechanism.

The electronic module may further comprise a communication unit for wireless communicating with another device, e.g., for wireless communication with the charger station and/or a mobile phone. The communication unit may comprise a wireless communication interface for communicating with another device via a wireless network such as Wi-Fi or Bluetooth, or even an interface for a wired communications link, such as a socket for receiving a Universal Serial Bus (USB), USB-C, mini-USB or micro-USB connector. Preferably, the electronic module comprises an RF, WiFi and/or Bluetooth unit as the communication unit. The communication unit may be provided as a communication interface between the electronic module and the exterior, such as other electronic devices, e.g., mobile phones, personal computers, laptops and so on. For example, dose data may be transmitted by the communication unit to the external device. The dose data may be used for a dose log or dose history established in the external device. In addition or as an alternative, the communication unit may receive data from the external device, e.g., data regarding a health condition of a user and/or dose data regarding an amount of drug to be delivered by the drug delivery device.

According to an independent aspect of the present disclosure, a charging station for charging an electronic encoder module for a drug delivery device is provided. This charging station may comprise a socket, a battery unit and electrical contacts, wherein said socket is configured to receive said electronic encoder module together with or without said drug delivery device. The electrical contacts of said charging station are arranged inside said socket permitting to electrically connect to the electrical contacts of the electronic encoder module if the electronic encoder module is to be charged. The battery unit is configured to provide the required power for charging. For example, when the electronic encoder module is electrically connected to the charging station for charging the battery of the electronic encoder module, said charging station and said electronic encoder module comprise means such as ribs provided inside said socket engaging with respective recesses of said electronic encoder module allowing only a predetermined orientation connecting in which charging is guaranteed.

A battery powered docking station or charging pen may comprise a battery, e.g., a rechargeable battery (accumulator) or a non-rechargeable lifetime battery, and spring contacts for contacting the electrical contacts of the electronic encoder module during charging of the module. Such a battery powered docking station or charging pen may further comprise means for indicating a status of the station and/or the module, e.g., at least one LED to display the charging status. Such stations have the advantage of being relatively inexpensive not requiring cables or interfaces.

According to a further independent aspect of the present disclosure, a method for charging the electronic encoder module with a charging station is provided. The method may comprise the following steps: connecting a charging station to a power source to charge a battery unit of said charging station; inserting a drug delivery device including an electronic encoder module into the charging station or removing the electronic encoder module from the drug delivery device and inserting said electronic encoder module into the charging station thereby electrically connecting the electrical contacts of said electronic encoder module with the electrical contacts of the charging station; removing the drug delivery device or the electronic encoder unit from the charging station. The first step of charging a battery unit of the charging station may be omitted if the charging station is provided with a lifetime battery not requiring charging.

According to a still further aspect of the present disclosure, a drug delivery device is provided which may comprise or may be coupled to an electronic module, wherein the electronic module is configured such that its rechargeable battery may be charged by means of electrical contacts.

The drug delivery device may be a reusable device permitting replacement of an empty cartridge. For example, the cartridge may be received in a releasably attached cartridge holder.

In one embodiment, the drug delivery device comprises a dial sleeve, e.g., a number sleeve, which is rotatable relative to a housing, e.g., along a helical path, at least in the dose setting operation. In addition, a manually operable injection trigger, for example a dose and/or injection button or a member axially and/or rotationally locked thereto may be axially displaceable relative to the dial sleeve and rotationally constrained to the housing at least in the dose delivery operation.

In an example, the drug delivery device may comprise an electronic encoder module, a housing and a dose setting and drive mechanism which is configured to perform a dose dialing operation for selecting a dose to be delivered by the drug delivery device and a dose delivery operation for delivering the set dose. The electronic encoder module may be detachably coupled to said drug delivery device. Detecting a dose dialing operation and/or a dose delivery operation may be achieved with the drug delivery device further comprising an encoder disk or encoder ring with a ring of teeth, wherein the electronic encoder module is coupled to the drug delivery device such that a movement of the encoder disk or encoder ring relative to the at least one sensor unit is detectable by the at least one sensor unit. In more detail, the encoder disk or encoder ring may comprise, e.g., twelve, light reflecting teeth spaced by 30° from each other, wherein the at least one sensor unit is arranged such that, depending on the rotational position of the encoder, light emitted by one of the light sources is reflected by one of the teeth and detected by the respective optical sensor, while light emitted by the other of the light sources is not reflected by one of the teeth and consequently not detected by the respective optical sensor. In one example, the encoder and the optical sensor units are in an anti-phase arrangement which means that if both light sources simultaneously emit light, only one of the optical sensors receives the light whereas the other optical sensor does not receive the emitted light. As the encoder and the sensor units are moved relative to each other, the optical sensor which previously received the light now does not receive the emitted light whereas the other optical sensor does receive the light. This may be achieved by the encoder disk or encoder ring selectively reflecting light. As an alternative, the encoder disk or encoder ring may selectively block light. In other examples the encoder disk or encoder ring and the optical sensor units are not in an anti-phase arrangement or arranged with a 90° phase difference, such that if both light sources simultaneously emit light, none or only one or all optical sensors detect the light depending on the relative position of the encoder.

The electronic system may be configured as a re-usable clip-on module for an injection device or may be permanently attached to a drug delivery device. For this purpose, the electronic encoder module may further comprise retention features for permanently or releasably attaching the module to a drug delivery device, for example to a dial grip or button of a drug delivery device. As an alternative, the electronic system may be a unit or module integrated (built in) into an injection device. The terms electronic system and (electronic) module are used synonymously in the following for both alternatives. The functionality of recording doses may be of value to a wide variety of device users as a memory aid or to support detailed logging of dose history. It is envisaged that the electronic system, e.g., an electronic module, could be configured to be connectable to a mobile phone, or similar, to enable the dose history to be downloaded from the system on a periodic basis.

The present disclosure further pertains to a drug delivery device with the electronic module, e.g., a dose recording module as disclosed in WO 2021/116387 A1, for use with a charging system as described above which drug delivery device comprises a cartridge containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω)-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g., the axis which extends through the proximal and distal ends of the cartridge, the cartridge holder or the drug delivery device.

### BRIEF DESCRIPTION OF THE DRAWINGS

Non-limiting, exemplary embodiments of the disclosure will now be described with reference to the accompanying drawings, in which:

FIGS. 1a, b show in a perspective view embodiments of a charging system according to the present invention;

FIGS. 2a-d show in a perspective view embodiments of an electronic module according to the present invention; and

FIG. 3 shows in a partially exploded view the module of FIG. 2a.

In the figures, identical elements, identically acting elements or elements of the same kind may be provided with the same reference numerals.

### DETAILED DESCRIPTION

In the following, some embodiments will be described with reference to an insulin injection device. The present disclosure is however not limited to such application and may equally well be deployed with injection devices that are configured to eject other medicaments or drug delivery devices in general, preferably pen-type devices and/or injection devices.

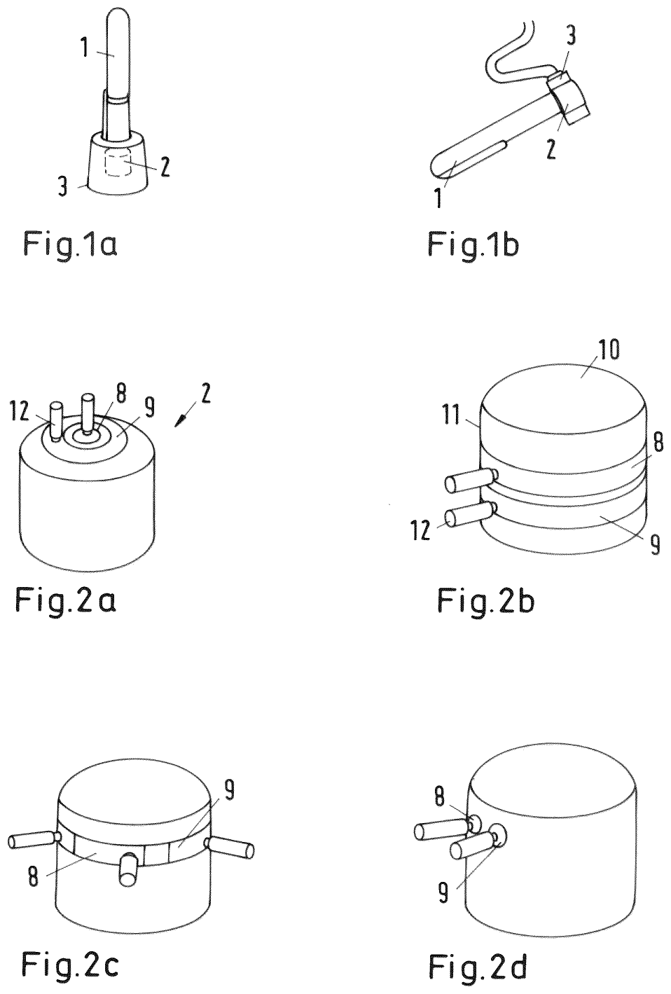

FIGS. 1a and 1b show a pen-type drug delivery device 1 with an electronic module 2 which may be attached to or integrated into the drug delivery device 1. In the depicted exemplary embodiment, the electronic module 2 is arranged at the proximal end, i.e., the end facing away from an injection needle, of the drug delivery device 1. The electronic module 2 may be fully integrated, e.g., in a button or knob of the drug delivery device 1, or may be permanently or releasably attached to the drug delivery device 1, e.g., as a cap connectable with a button or knob of the drug delivery device 1.

In addition, a charger station 3 is depicted connected to the module 2, wherein FIG. 1a schematically shows a charger station suitable to hold and partially receive the module 2 and/or the drug delivery device 1, whereas FIG. 1b schematically shows a cap-like charger station which may be fitted onto the module 2 for charging.

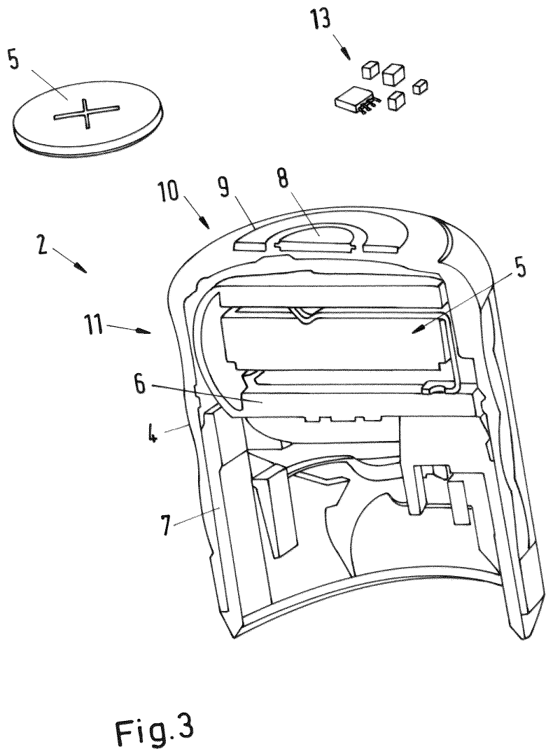

As shown in FIGS. 2a to 3 in more detail, the electronic encoder module 2 comprises an outer shell 4 housing or encasing at least partially a rechargeable battery (accumulator) 5 and a PCBA (printed circuit board assembly) 6 connected to the battery 5. The battery 5 and the PCBA 6 may be held in a chassis 7 retained in the outer shell 4 (FIG. 3).

The module 2 may further comprise at least one sensor unit (not depicted), for example consisting of at least two light sources, e.g., IR-LEDs, and at least two optical sensors, e.g., light detectors. The sensor unit is configured for detecting a movement of an, e.g., rotatable, encoder element, like a disk or ring, of a drug delivery device if the module 2 is attached to the drug delivery device 1. Typically, this detectable movement is indicative of doses that are dialed and/or delivered from the drug delivery device 1. Still further, the module 2 may comprise a communication unit (not depicted), e.g., for wireless communication with another device, like a smart phone, a network or a PC. The PCBA 6 may comprise a processor configured to control operation of the at least one sensor unit and to process and/or store signals from the at least one sensor unit.

The battery 5 may be a lithium-ion battery having a capacity of, e.g., about 25 mAh at 3.7V with a peak current of, e.g., about 50 mA. Such a battery is suitable for use in a module 2 with a lifetime of, e.g., about 15 to about 20 weeks without recharging. As an alternative, the battery 5 may be a NiMH battery having a capacity of, e.g., about 6 mAh at 1.2V with a peak current of, e.g., about 18 mA. The NiMH battery has the advantage of a relatively small height of less than 2.5 mm, e.g., about 2.3 mm. Such a battery is suitable for use in a module 2 with a lifetime of, e.g., 2 weeks without recharging.

To allow simple and reliable recharging of the battery 5, the module 2 is provided with two electrical contacts 8, 9 connected directly to the battery 5 or indirectly via the PCBA 6. As shown in FIG. 2a, the contacts may be provided on the proximally arranged top surface 10 of the outer shell 4, e.g., as a central contact area 8 concentrically surrounded by a ring-shaped contact 9. In FIG. 2b, the electrical contacts 8, 9 are provided as two rings arranged on the lateral surface 11 of the outer shell 4. A further alternative is depicted in FIG. 2c, wherein the contacts 8, 9 are provided as ring segments on the lateral surface 11 of the outer shell 4. Still further, FIG. 2d depicts the contacts as two contact areas on the lateral surface 11 of the outer shell 4.

The respective electrical contacts 8, 9 are made from an electrically conductive material and are surrounded by a non-conductive material of the outer shell 4. The electrical contacts 8, 9 may be attached to the outer shell 4, e.g., by gluing, by press fit and/or by overmolding. Charging of the battery 5 is effected by establishing contact between the electrical contacts 8, 9 and spring contacts 12 of the charger station 3. Such spring-loaded contacts are a good and robust solution. However other configurations, e.g., elastic contacts made from spring-type sheet metal, are suitable, too.

In FIG. 3, an optional boost supply unit 13 is schematically indicated which may be retained in chassis 7. The boost supply unit 13 may be arranged on or may be part of the PCBA 6. The boost supply unit 13 is configured to provide a constant equal voltage, for example 3.3 V, from a battery having a lower voltage of, e.g., 1.2V.

The drug delivery device 1 may be based on the same general working principle as the disposable injection pen disclosed in EP 2 890 434 B1 to which reference is made regarding the main functions and the operation modes. A non-limiting example of a module 2 is disclosed in WO 2021/116387 A1 and PCT/EP2021/060631 to which reference is made regarding the main functions and the operation modes of an electronic button module.

### REFERENCE NUMERALS

1 drug delivery device
2 electronic module
3 charger station
4 outer shell
5 battery
6 PCBA
7 chassis
8 electrical contact
9 electrical contact
10 top surface
11 lateral surface
12 spring contact
13 boost supply unit

1. An electronic encoder module for use with a drug delivery device, the electronic encoder module comprising:
at least one sensor unit;
a PCBA with a processor configured to control operation of the at least one sensor unit and to process and/or store signals from the at least one sensor unit;
a rechargeable battery connected to the PCBA; and
a cap-shaped outer shell at least partially encasing the at least one sensor unit, the PCBA and the rechargeable battery, wherein the cap-shaped outer shell comprises a lateral surface and a proximally arranged top surface, wherein at least two electrical contacts connected to the rechargeable battery and/or the PCBA are provided on the lateral surface and/or on the proximally arranged top surface.

2. The electronic encoder module according to claim 1, wherein said electrical contacts comprise at least one ring, at least one ring segment and/or at least one contact area made from an electrically conductive material and are surrounded by a non-conductive material of the cap-shaped outer shell.

3. The electronic encoder module according to claim 2, wherein the electrical contacts are both arranged on the lateral surface of the cap-shaped outer shell.

4. The electronic encoder module according to claim 2, wherein the electrical contacts are both arranged on the proximally arranged top surface of the cap-shaped outer shell.

5. The electronic encoder module according to claim 1, wherein the electrical contacts are attached to the cap-shaped outer shell by gluing, by press fit and/or by overmolding.

6. The electronic encoder module according to claim 1, further comprising a boost supply unit configured to provide a constant equal voltage.

7. The electronic encoder module according to claim 1, wherein said rechargeable battery is a coin cell with a charging capacity between 6 mAh and 25 mAh and/or a height in between 1.5 mm to 4.5 mm.

8. The electronic encoder module according to claim 1, further comprising a communication unit for wirelessly communicating with another device.

9. The electronic encoder module according to claim 1, wherein said at least one sensor unit comprises at least two light sources and at least two optical sensors, wherein said at least one sensor unit is configured for detecting a movement of an encoder of the drug delivery device if the electronic encoder module is attached to the drug delivery device, wherein the movement is indicative of doses that are dialed and/or delivered from the drug delivery device.

10. The electronic encoder module according to claim 1, further comprising retention features for permanently or releasably attaching the electronic encoder module to the drug delivery device.

11. A drug delivery device comprising an electronic encoder module, wherein said drug delivery device comprises at least a housing, a dose setting and drive mechanism which is configured to perform a dose dialing operation for selecting a dose to be delivered by the drug delivery device and a dose delivery operation for delivering the set dose, the electronic encoder module comprising:
at least one sensor unit;
a PCBA with a processor configured to control operation of the at least one sensor unit and to process and/or store signals from the at least one sensor unit;
a rechargeable battery connected to the PCBA; and
a cap-shaped outer shell at least partially encasing the at least one sensor unit, the PCBA and the rechargeable battery, wherein the cap-shaped outer shell comprises a lateral surface and a proximally arranged top surface, wherein at least two electrical contacts connected to the rechargeable battery and/or the PCBA are provided on the lateral surface and/or on the proximally arranged top surface.

12. The drug delivery device according to claim 11, wherein said electronic encoder module is detachably coupled to said drug delivery device.

13. The drug delivery device according to claim 11, further comprising an encoder with a ring of teeth, wherein the electronic encoder module is coupled to the drug delivery device such that a movement of the encoder relative to the at least one sensor unit is detectable by the at least one sensor unit.

14. (canceled)

15. The drug delivery device according to claim 11, further comprising a cartridge containing a medicament.

16. The electronic encoder module according to claim 9, wherein the at least two light sources comprise at least one IR-LED or the at least two optical sensors comprise a light detector.

17. The electronic encoder module according to claim 10, wherein the retention features attach to a dial grip or button of the drug delivery device.

18. The drug delivery device according to claim 13, wherein said at least one sensor unit comprises at least two light sources and at least two optical sensors, wherein said at least one sensor unit is configured for detecting the movement of the encoder if the electronic encoder module is attached to the drug delivery device, wherein the movement is indicative of doses that are dialed and/or delivered from the drug delivery device.

19. The drug delivery device according to claim 18, wherein the encoder comprises a plurality of light reflecting teeth spaced by 30° from each other, and wherein the at least one sensor unit is arranged such that, depending on a rotational position of the encoder, light emitted by one of the at least two light sources is reflected by one of the light reflecting teeth and detected by a respective optical sensor, while light emitted by the other of the at least two light sources is not reflected by one of the light reflecting teeth.
